(19) United States
(12) Patent Application Publication
(10) Pub. No.: US 2008/0257362 A1
(43) Pub. Date: Oct. 23, 2008

(54) Optically corrected soft eyewear for protection and hydration of eyes

(76) Inventors: Suzanne Davison, Temecula, CA (US); Roy Victor Paulson, Temecula, CA (US)

Correspondence Address: DONN K. HARMS;PATENT & TRADEMARK LAW CENTER, SUITE 100, 12702 VIA CORTINA, DEL MAR, CA, 92014, US

(21) Appl. No.: 12/148,335

(22) Filed: Apr. 18, 2008

Related U.S. Application Data
(60) Provisional application No. 60/925,408, filed on Apr. 19, 2007.

(51) Int. Cl. A61F 9/04

(52) U.S. Cl. 128/858

(57) ABSTRACT

An eyewear apparatus which maintains a sealed cavity in front of a user's eyes when engaged to the face in an as-worn position. The device features a pair of eyecups formed of a soft pliable material and having an optically corrected curved lens portion formed of a soft pliable material. A projecting edge portion surrounding said curved lens provides a seal to maintain the cavity between the lens and the user's eye sealed from contaminants such a pollen and dirt and maintains humidity higher in the cavity for eye comfort.

This application claims the benefit of U.S. Provisional Application No. 60/925,408 filed on Apr. 19, 2007 and incorporated herein in its entirety by reference.

### FIELD OF THE INVENTION

This invention relates generally to an apparatus, system and method for employing soft, optically corrected lenses in a sealed engagement over the user's eyes for protecting the eyes from dirt, dust, and other contaminants, as well as maintaining humidity of the eye for hydration. The device can be worn in sealed engagement with the user's eyes, behind corrective eyewear or protective eyewear, such as eyeglasses, goggles, sunglasses, and the like, or may also be worn alone.

### BACKGROUND OF THE INVENTION

Approximately 60 million Americans suffer from dry eye conditions alone. Of these, approximately 10 million Americans have been diagnosed with severe dry eye conditions, e.g., Dry Eye Syndrome or Chronic Dry Eye Disease (CDED), both clinically known as keratoconjuctivitis sicca. CDED is a condition that results from inflammation of the eye, as well as inflammation of the tear-producing glands. This inflammation decreases the eyes' ability to produce natural tears, which help protect the eyes from irritation and keep them moisturized and lubricated.

Still further, many people suffer dry eyes while outdoors, or need protection from particulate such as dirt, dust, pollen, and the like, that can enter the eyes in windy, soiled areas. Additionally, many people with normal hydration need something to maintain it when in cold dry climates such as when skiing, or in hot dry climates such as the desert. The disclosed device works especially well for this purpose since it can be worn behind conventional glasses and goggles in combination therewith.

The invention accordingly comprises the features of construction, combination of elements, and arrangement of parts which will be exemplified by the constructions hereinafter set forth, and the scope of the invention will be indicated in the claims.

### SUMMARY OF THE INVENTION

The disclosed device features an eyewear device for maintaining the eyes in a hydrated condition while concurrently providing an optically correct, or corrected view through the device for the user. Unlike previous lenses which are generally hard plastic if optically correct, the disclosed device employs soft, optically corrected, substantially transparent cups, each having a sealing perimeter edge portion adapted to engage the orbital socket around the eye. The playable edge is planar comparted to the curved optic, and projects from the perimeter termination of the lens portion of the device thus providing a larger surface to achieve a better seal around an eye.

The cups being optically correct in the curved lens portion inside the projecting edge portion, can unlike other eye sealing devices, be concurrently behind other eyewear such as corrective glasses, or sunglasses, or goggles. This provides the user great utility to protect their eyes from irritation and harm in dusty or pollen infested areas. When engaged to the face in the as-worn position, the clear, soft, pliable, optically correct material forming both the lens and the projecting edge, will maintain pollutants outside the eye, and moisture sealed inside.

The optically correct cups, formed in a unibody construction, may be uncoated and yield excellent results with a sealed condition of the eye. But it is preferred to include an abrasion resistant exterior coating of thermoset clear material such as thermoset polyurethane, on the exterior and additionally an anti fog coating to prevent fogging in the sealed interior cavity.

A thermoset exterior coating layer, is also preferred in that it provides a self-healing surface to the optic portion of the cup in case of scratches to the exterior surface.

Optionally, the material forming the two cups may also be ultraviolet absorbent to thereby protect the device from clouding over time, and to also protect the eyes of the user from radiation such as sunlight. Laser absorbers may be added to the extruded material forming the device to protect against laser light encountered by the military.

The cups providing the lens and the seal, are formed in toroidal surfaces in the current best mode for optical correctness and an optimal flex modulus of each cup of the device. Preferred materials for the thermoplastic portion are of exceptional clarity when cured and include polyurethane, Ionomer, PVC, and Polyester. For the thermoset portion of the device polyurethane and silicone work especially well.

A unique flexible seal portion projecting and surrounding the lens or optic portion of the cup projects at an angle from the perimeter of the lens portion to achieve a complete seal to the face at the eye socket. The seal portion is formed of soft resilient material which minimizes pressure which can normally reduce localized blood flow and thereby maintains user comfort.

In an as-worn position, with the flexible seal in contact with the user's face surrounding their eye and forming a seal therewith, the product forms an interior cavity in-between the cup and the eye. This interior cavity has an increased humidity and temperature to comfort the eye and provides comfort from low humidity, low temperature, wind, dust, pollen, and projectiles.

The flexible lens portion of the cup, is preferred to incorporate an anti-fog coating on the inside wall facing the eye in the as-worn position. The exterior surface, opposite the inside wall, preferably employs an abrasion resistant coating for durability.

In use to position the two cups in the as-worn position, in sealed or semi-sealed engagement to the face of the user and covering each eye, a means for positioning of the projecting planar edge to hold the edge in sealed engagement to the face surrounding the eye of the user is employed with the device. Such a means for positioning may be temples, an elastic band if a biased engagement of the planar edge is desired, or, the cups might be attached directly to other glasses at the temples and bridge and achieve a seal when the glasses or mounted on the face, or combinations of the above may be used.

Additionally, the clear flexible lens in the as-worn position is optically corrected, as such, the user may be fitted for prescription lenses and submit to an eye exam while wearing the product.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the presently disclosed device employing soft optically corrected toroidal shaped lenses with surrounding seals. It is important, therefore, that the claims be regarded as including such equivalent construction and methodology insofar as they do not depart from the spirit and scope of the present invention.

It is an object of this invention to provide a soft optically corrected lens which may be worn behind and in combination with other eyewear such as sunglasses or prescription glasses.

It is an additional object of this invention to provide such a soft lens which achieves a seal with the face surrounding the eye and thereby forms a cavity between the eye and the lens to keep moisture in and particulate out.

These together with other objects and advantages which become subsequently apparent reside in the details of the construction and operation as more fully hereinafter described and claimed, reference being had to the accompanying drawings forming a part thereof, wherein like numerals refer to like parts throughout.

### BRIEF DESCRIPTION OF THE DRAWING FIGURES

In the drawing figures, which are not to scale and which are merely illustrative and wherein like reference characters denote similar elements throughout the several views:

FIG. 1 is a front perspective view of an eyewear apparatus formed of two clear, optically corrected lenses, soft flexible material and having a perimeter edge portion projecting at an angle adapted to seal within the orbital bone around the eye. A flexible strap holds the device in the as-worn position.

FIG. 2 is a front perspective view of the device herein which employs flexible temples as a means to maintain the device in an as-worn position.

FIG. 3 is a rear view of the clear optically correct eye cups showing the two eye chambers that will maintain eye hydration when worn and prevent dust and particulate from entering the eye.

FIG. 4 is a sliced view through two adjacent lenses showing the projecting seal from the perimeter of each lens portion.

FIG. 5 is a sliced view showing the projecting edge adapted for orbital seal and the chamber formed by the curved, clear, optically correct material forming the soft eye cups and optional coatings.

### DETAILED DESCRIPTION OF THE EXEMPLARY EMBODIMENTS

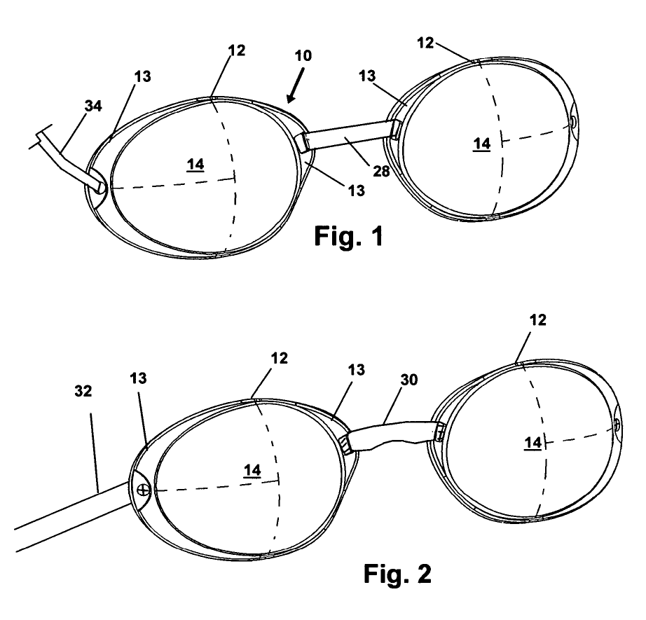

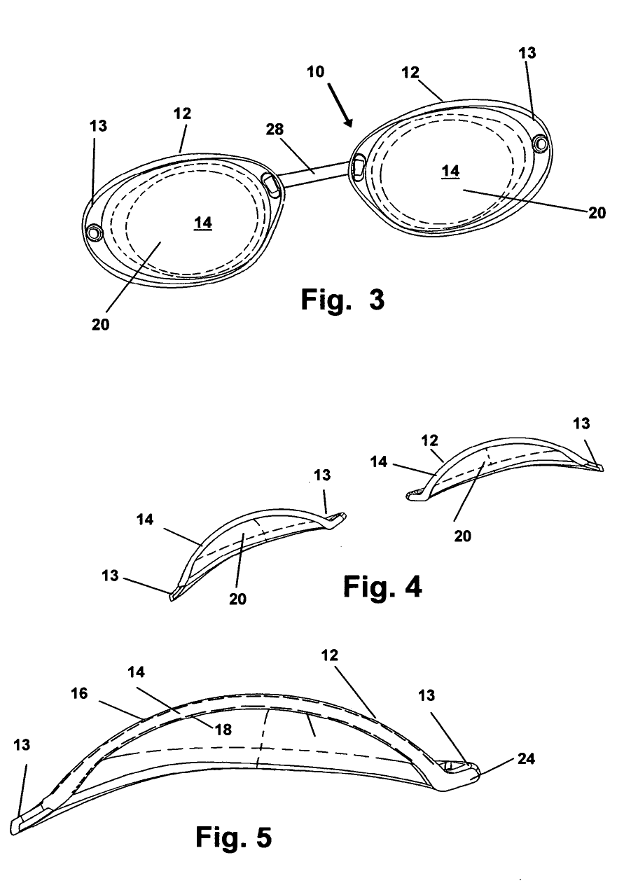

Referring now to the drawings of FIGS. 1-5 show the various aspects of the device 10 as employed in the as-worn position with one or two cups 12 formed to seal with the face of a user in the area surrounding the eye. The seal is provided by the projecting pliable edge 13 which surrounds the lens 14 of each cup 12.

The cups 12 have a lens 14 portion which is soft, optically corrected and substantially transparent. The substantially planar pliable edge 13 projects from the perimeter termination of the lens 12 portion of the device 10 and thereby provides a larger surface area to achieve a better seal around an eye socket of the user. The projecting edge 13 portion is soft resilient material like the optic or lens 12 portion. This soft material, combined with the planar area and angle adapted to engage the eye socket, yield an especially good sealing area around the orbital bone for each cup 12.

The cups 12 being optically correct in the curved lens 14 portion inside the projecting substantially planar edge 13 portion, can be worn concurrently behind other eyewear such as corrective glasses, or sunglasses, or goggles. As noted, this makes them especially useful for users in dusty or pollen infested areas since the clear, soft, pliable, optically correct material forming both the lens and the projecting edge 13, will maintain pollutants outside the eye, and moisture sealed inside the chamber adjacent to the eye. As noted, the device 10 allows users seeking to maintain their eye moisture at high altitudes, or dry climates, or seeking to keep particulate and pollen from their eyes, but wanting to wear sunglasses or corrective lenses by allowing such eyewear to be worn concurrently with the device 10. This alleviates the need for such users who must or want to use their expensive or prescription eyewear from suffering from climate, allergies, and particulate.

The optically correct lens 14 in each cup 12, formed preferably in a unibody construction, may be uncoated and provide substantial improvement to users. However, it is preferred to include an abrasion resistant thermoset exterior coating 16 of thermoset clear material such as polyurethane or silicone. Additionally it may be preferable to also include an anti fog coating 18 to prevent fogging in the sealed interior cavity 20 formed by the curved lens 14 portion, the eye, and the face of the user.

Preferably, the wall of the interior material of the cup, closest to the eye and face, is thermoplastic material 24 which is softer than the exterior wall surface on the opposite side of the cup providing cushioning for use over long periods. The thermoset exterior coating 16 is also preferred in that it provides a self-healing surface to the optic or lens 14 portion of the cup 12 in case of scratches to the exterior surface.

The material forming the two cups, being clear, is also ultraviolet absorbent to protect both the product from clouding over time, and to protect the eyes of the user from radiation such as sunlight. Laser absorbers may be added to the extruded material forming the device which would match the wave lengths of lasers most likely to be encountered and would darken instantly should the lens be struck by a laser such as a laser pointer currently in the red light wave spectrum and widely used and abused.

The absorbers may also be included to block other wavelengths of light as the case may be should the devices for instance be employed in a war zone where laser weapons are a reality. The fact that each cup 12 is independently engaged to the face and can be replaced if damaged, and maintains the eyes free of dust and particulate, yet moist, make the device particularly useful for the military when worn behind sunglasses or other lenses to aid in keeping the eyes healthy and protect them from lasers and particulate and projectiles from nearby blasts or high winds.

Since the cups 12 are independently engageable to the strap 28 or bridge 30, and each has the soft planar edge portion projecting from the circumference of the curved optic or lens 14, they may easily be replaced if damaged by projectiles, or lasers.

The cups 12 have the lens 14 formed in a substantially toroidal surface in the current best mode for optical correctness, and flex modulus of each cup 12 and lens 14 of the device 10.

As noted, the device 10 provides exceptional utility since each disclosed cup 12 incorporates a clear, optically corrected lens 14 portion for use with and without prescription glasses or sunglasses. Surrounding this lens 12 a substantially planar flexible seal portion 13 employs a combination of shape, planar area, and flexibility and angle away from the perimeter edge of the lens 12 to achieve an excellent seal.

The outside edge of the projecting planar portion providing the seal may optionally be engaged with a thin foam seal or gasket if desired. Should some air flow into the cavity 20 formed by the device 10 and eye of the user be desired, an open cell foam may be used. This will provide a means for filtered air flow into the cavity 20 formed between the cup 12 and the eye of the user.

This unique flexible seal 13 portion projecting and surrounding the lens 14 or optic portion of the cup 12, allows the device to achieve a complete seal to the face. However, the soft resilient material forming the device minimizes pressure which can normally reduce localized blood flow and thereby maintains user comfort.

The lens 14 formed in the cup 12 may be molded with photo-chromic dyes for automatic darkening of the lens 14 when in sunlight. Alternatively, a coating may be employed of photo-chromic dyes. The photo-chromic medium, whether coated or included in the material at formation, may also be adapted to darken in response to a laser pulse.

In use to position the two cups 12 in the as-worn position, in sealed or semi-sealed engagement to the face of the user and covering each eye, a means for positioning of the projecting planar edge 13 to hold the edge in sealed engagement to the face surrounding the eye of the user is employed with the device 10. This means for positioning the lens design could may be temples 32, an elastic band 34 if a biased engagement of the edge 13 is desired. The cups 12 might be attached directly to other glasses at the temples and bridge and achieve a seal when the glasses or mounted on the face.

The projecting edge 13 portions are formed of flexible material, as is the lens 14 and consequently the potential for scratching the inner lens surface of other glasses worn concurrently with the device 10 is virtually eliminated since the lens 14 portion will simply deform toward the user's eye should it contact a pair of hard lenses in glasses. An additional benefit from the flexible lens 14 design which abuts the face at its base engagement toe the projecting edge 13, is that the toroidal shape maximizes the peripheral viewing field of the wearer.

If needed, the two cups forming the device, and their respective projecting seals, may be individual or unitary in construction. In individual mode, elastic straps, or other means to hold the lenses in position would be employed. In the singular mode, the device might appear somewhat like a clear mask with two toroidal surfaces forming projecting cavities in front of each eye. Or, the formed lenses might be singular and attached with a bridge over the nose, or lens can be singular and attached and or secured with an existing bridge. As noted, in all modes of the device, the lenses may be formed in colors, and may include photo chromic properties.

While all of the fundamental characteristics and features of the device herein have been disclosed and described, with reference to particular embodiments thereof, a latitude of modification, various changes and substitutions are intended in the foregoing disclosure and it will be apparent that in some instance, some features of the invention will be employed without a corresponding use of other features without departing from the scope of the invention as set forth. It should be understood that such substitutions, modifications, and variations may be made by those skilled in the art without departing from the spirit or scope of the invention. Consequently, all such modifications and variations are included within the scope of the invention as defined herein.

What is claimed is:

1. An eyewear apparatus for maintaining a sealed cavity in front of a user's eyes when engaged to the face in an as-worn position, comprising:
a pair of eyecups, each said eyecup having a soft, pliable, curved lens portion extending to a circumferential edge;
each eyecup having projecting edge portion formed of flexible material, extending from said circumferential edge, said projecting edge portion having a first surface positionable in contact with said face of said user in a seal, when in said as-worn position;
said sealed cavity defined by the area between an interior surface of said curved lens portion and said eye and said face of said user surrounding said eye, when said eyecups are in said as-worn position;
said lens portion being optically corrected;
said lens portion deformable into said cavity by pressure thereon from outside said cavity;
said eyewear apparatus wearable concurrently with prescription or non-prescription eyeglasses by said user without effecting the vision of said user through said eyeglasses;
said sealed cavity providing means to maintain humidity higher in said cavity an means to prevent particulate from contacting said eye; and
said lens portion deformable into said cavity should a contact occur with a lens of said eyeglasses thereby providing means to prevent damage to said eyeglasses from contact with a said lens.

2. The eyewear apparatus of claim 1, additionally comprising:
said first surface positionable said projecting edge portion being substantially planar thereby providing means to increase a contact area of said seal.

3. The eyewear apparatus of claim 1, additionally comprising:
an anti-fog coating on said interior surface of said curved lens portion.

4. The eyewear apparatus of claim 1, additionally comprising:
said curved lens portion having an exterior surface opposite said interior surface;
a thermoset exterior coating on said exterior surface providing means to prevent scratches on said exterior surface and means for self healing of said exterior surface.

5. The eyewear apparatus of claim 2, additionally comprising:
said curved lens portion having an exterior surface opposite said interior surface;
a thermoset exterior coating on said exterior surface providing means to prevent scratches on said exterior surface and means for self healing of said exterior surface.

6. The eyewear apparatus of claim 3, additionally comprising:
said curved lens portion having an exterior surface opposite said interior surface;
a thermoset exterior coating on said exterior surface providing means to prevent scratches on said exterior surface and means for self healing of said exterior surface.

7. The eyewear apparatus of claim 2, additionally comprising:
an anti-fog coating on said interior surface of said curved lens portion.

8. The eyewear apparatus of claim 1, additionally comprising:
means for biased engagement of said first surface of said projecting edge portion in contact with said face of said user.

9. The eyewear apparatus of claim 2, additionally comprising:
means for biased engagement of said first surface of said projecting edge portion in contact with said face of said user.

10. The eyewear apparatus of claim 3, additionally comprising:
means for biased engagement of said first surface of said projecting edge portion in contact with said face of said user.

11. The eyewear apparatus of claim 4, additionally comprising:
means for biased engagement of said first surface of said projecting edge portion in contact with said face of said user.

12. The eyewear apparatus of claim 5, additionally comprising:
means for biased engagement of said first surface of said projecting edge portion in contact with said face of said user.

13. The eyewear apparatus of claim 6, additionally comprising:
means for biased engagement of said first surface of said projecting edge portion in contact with said face of said user.

14. The eyewear apparatus of claim 1, additionally comprising:
said projecting edge having a second surface, opposite said first surface;
said first surface formed of thermoplastic material which is softer than said second surface;
said first surface thereby providing means for cushioning for increased comfort of said user when said eyewear apparatus is in said as-worn position for long time periods.

15. The eyewear apparatus of claim 2, additionally comprising:
said projecting edge having a second surface, opposite said first surface;
said first surface formed of thermoplastic material which is softer than said second surface;
said first surface thereby providing means for cushioning for increased comfort of said user when said eyewear apparatus is in said as-worn position for long time periods.

16. The eyewear apparatus of claim 6, additionally comprising:
said projecting edge having a second surface, opposite said first surface;
said first surface formed of thermoplastic material which is softer than said second surface;
said first surface thereby providing means for cushioning for increased comfort of said user when said eyewear apparatus is in said as-worn position for long time periods.

17. The eyewear apparatus of claim 13, additionally comprising:
said projecting edge having a second surface, opposite said first surface;
said first surface formed of thermoplastic material which is softer than said second surface;
said first surface thereby providing means for cushioning for increased comfort of said user when said eyewear apparatus is in said as-worn position for long time periods.

18. The eyewear apparatus of claim 1, additionally comprising:
said curved lens portion including a photo-chromic dye thereby providing means for automatic darkening of the lens when in sunlight.

19. The eyewear apparatus of claim 17, additionally comprising:
said curved lens portion including a photo-chromic dye thereby providing means for automatic darkening of the lens when in sunlight.
